(19) United States
(12) Patent Application Publication
(10) Pub. No.: US 2015/0166425 A1
(43) Pub. Date: Jun. 18, 2015

(54) PROCESS FOR OLIGOMERIZING GASOLINE WITH HIGH YIELD

(71) Applicant: UOP LLC, Des Plaines, IL (US)

(72) Inventors: Steven L. Krupa, Fox River Grove, IL (US); Todd M. Kruse, Oak Park, IL (US); Kurt M. Vanden Bussche, Lake in the Hills, IL (US); Christopher P. Nicholas, Evanston, IL (US)

(73) Assignee: UOP LLC, Des Plaines, IL (US)

(21) Appl. No.: 14/108,594

(22) Filed: Dec. 17, 2013

(51) Int. Cl. C07C 2/08

(52) U.S. Cl. CPC C07C 2/08

(57) ABSTRACT

An olefinic feed stream comprising butenes is oligomerized over a zeolitic catalyst to make gasoline. Conversion of normal butenes is kept low to maximize gasoline production. Recycle of unconverted butenes provides for sufficient overall conversion to gasoline product.

### FIELD

The field of the invention is the oligomerization of light olefins to gasoline.

### BACKGROUND

When oligomerizing light olefins within a refinery, there is frequently a desire to make high octane gasoline which is highly branched. Catalysts that make high octane gasoline typically make product that is highly branched and within the gasoline boiling point range. In addition, zeolitic catalysts that make high cetane diesel typically make product that is more linear and in the distillate boiling point range. This results in less and poorer quality gasoline due to the more linear nature of the product which has a lower octane value.

The oligomerization of butenes is often associated with a desire to make a high yield of high quality gasoline product. There is typically a limit as to what can be achieved when oligomerizing butenes. When oligomerizing butenes, dimerization is desired to obtain gasoline range material. However, trimerization and higher oligomerization can occur which can produce material heavier than gasoline such as diesel.

When oligomerizing olefins, there is often a desire to maintain a liquid phase within the oligomerization reactors. A liquid phase helps with catalyst stability by acting as a solvent to wash the catalyst of heavier species produced. In addition, the liquid phase provides a higher concentration of olefins to the catalyst surface to achieve a higher catalyst activity. Typically, this liquid phase in the reactor is maintained by hydrogenating some of the heavy olefinic product and recycling this paraffinic product to the reactor inlet

It would be desirable to make high quality gasoline from a zeolitic catalyst.

### SUMMARY OF THE INVENTION

Zeolitic catalyst is used to oligomerize more highly branched gasoline range molecules by operating an oligomerization reactor at a low conversion per pass and a high recycle rate to achieve a high yield of gasoline product. In order to produce a high yield of gasoline product, a conversion per pass of no more than 50% is required at a combined feed ratio (CFR) of at least 2.0 to achieve a high overall feed olefin conversion. Two reactors may be used to accommodate a higher CFR to support the high overall feed flow to achieve the per pass conversion. A high space velocity and high reactor temperature may be employed to aid in the back-cracking of heavy oligomers produced.

An embodiment is a process for making gasoline comprising feeding an oligomerization feed stream and a recycle stream comprising butenes to an oligomerization zone; and oligomerizing butenes in the oligomerization feed stream and the recycle stream to butene oligomers over a zeolitic catalyst with a combined feed ratio of at least 2 and an n-butene conversion of no more than 50% wherein a selectivity to C₈-C₁₁ olefins is at least about 50%.

### BRIEF DESCRIPTION OF THE DRAWINGS

FIG. 1 is a schematic drawing of the present invention.

FIG. 2 is a plot of C₈-C₁₁ olefin selectivity versus normal butene conversion.

FIG. 3 is a plot of C₁₂+ olefin selectivity versus normal butene conversion.

### DEFINITIONS

As used herein, the term “stream” can include various hydrocarbon molecules and other substances. Moreover, the term “stream comprising Cₓ hydrocarbons” or “stream comprising Cₓ olefins” can include a stream comprising hydrocarbon or olefin molecules, respectively, with “x” number of carbon atoms, suitably a stream with a majority of hydrocarbons or olefins, respectively, with “x” number of carbon atoms and preferably a stream with at least 75 wt % hydrocarbons or olefin molecules, respectively, with “x” number of carbon atoms. Moreover, the term “stream comprising Cₓ+ hydrocarbons” or “stream comprising Cₓ+ olefins” can include a stream comprising a majority of hydrocarbon or olefin molecules, respectively, with more than or equal to “x” carbon atoms and suitably less than 10 wt % and preferably less than 1 wt % hydrocarbon or olefin molecules, respectively, with x−1 carbon atoms. Lastly, the term “Cₓ-stream” can include a stream comprising a majority of hydrocarbon or olefin molecules, respectively, with less than or equal to “x” carbon atoms and suitably less than 10 wt % and preferably less than 1 wt % hydrocarbon or olefin molecules, respectively, with x+1 carbon atoms.

As used herein, the term “zone” can refer to an area including one or more equipment items and/or one or more sub-zones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “substantially” can mean an amount of at least generally about 70%, preferably about 80%, and optimally about 90%, by weight, of a compound or class of compounds in a stream.

As used herein, the term “gasoline” can include hydrocarbons having a boiling point temperature in the range of about 25° to about 200° C. at atmospheric pressure.

As used herein, the term “diesel” or “distillate” can include hydrocarbons having a boiling point temperature in the range of about 150° to about 400° C. and preferably about 200° to about 400° C.

As used herein, the term “vacuum gas oil” (VGO) can include hydrocarbons having a boiling temperature in the range of from 343° to 552° C.

As used herein, the term “vapor” can mean a gas or a dispersion that may include or consist of one or more hydrocarbons.

As used herein, the term “overhead stream” can mean a stream withdrawn at or near a top of a vessel, such as a column.

As used herein, the term “bottom stream” can mean a stream withdrawn at or near a bottom of a vessel, such as a column.

As depicted, process flow lines in the figures can be referred to interchangeably as, e.g., lines, pipes, feeds, gases, products, discharges, parts, portions, or streams.

As used herein, “bypassing” with respect to a vessel or zone means that a stream does not pass through the zone or vessel bypassed although it may pass through a vessel or zone that is not designated as bypassed.

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottom stream back to the bottom of the column. Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column.

As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, “taking a stream from” means that some or all of the original stream is taken.

As used herein, a “combined feed ratio” (CFR) means the ratio of the sum of flow rates of fresh feed and recycled feed to the flow rate of just the fresh feed.

### DETAILED DESCRIPTION

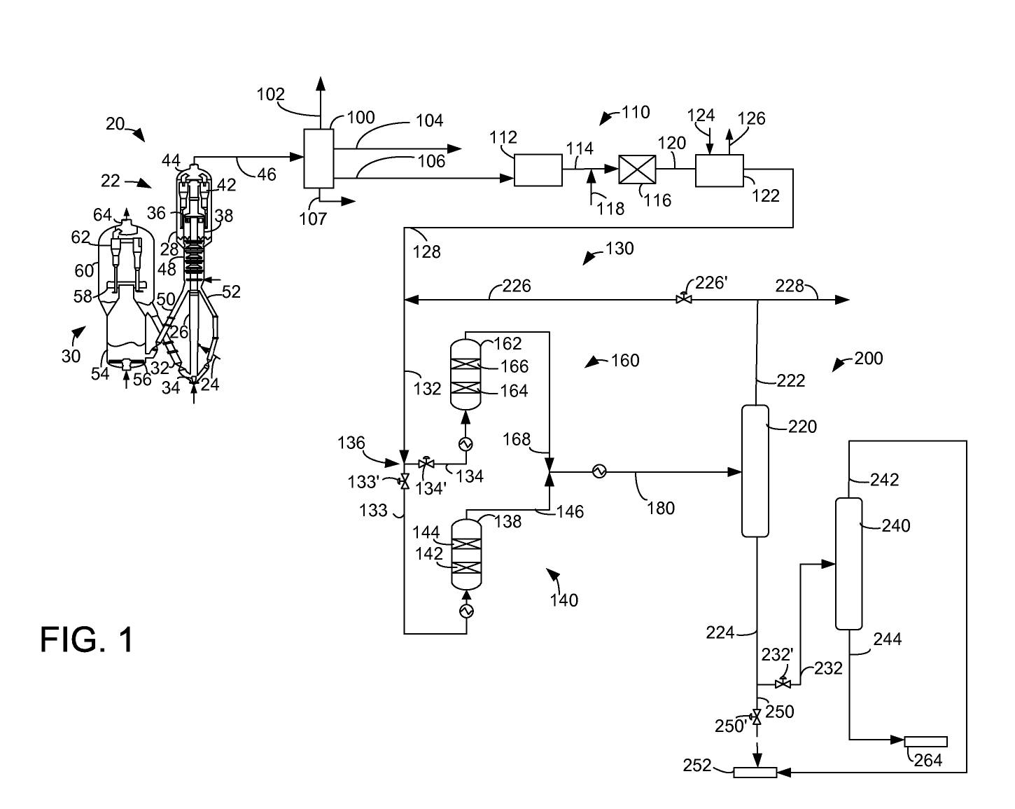

The present invention is an apparatus and process that can be used to primarily make gasoline and to make diesel. The apparatus and process may be described with reference to five components shown in FIG. 1: a fluid catalytic cracking (FCC) zone 20, an FCC recovery zone 100, a purification zone 110, an oligomerization zone 130, and an oligomerization recovery zone 200. Many configurations of the present invention are possible, but specific embodiments are presented herein by way of example. All other possible embodiments for carrying out the present invention are considered within the scope of the present invention.

The FCC zone 20 may comprise an FCC reactor 22 and a regenerator vessel 30.

A conventional FCC feedstock and higher boiling hydrocarbon feedstock are a suitable FCC hydrocarbon feed 24 to the FCC reactor. The most common of such conventional feedstocks is a VGO. Higher boiling hydrocarbon feedstocks to which this invention may be applied include heavy bottom from crude oil, heavy bitumen crude oil, shale oil, tar sand extract, deasphalted residue, products from coal liquefaction, atmospheric and vacuum reduced crudes and mixtures thereof.

The FCC reactor 22 may include a reactor riser 26 and a reactor vessel 28. A regenerator catalyst pipe 32 delivers regenerated catalyst from the regenerator vessel 30 to the reactor riser 26. A fluidization medium such as steam from a distributor 34 urges a stream of regenerated catalyst upwardly through the reactor riser 26. At least one feed distributor injects the hydrocarbon feed in a hydrocarbon feed line 24, preferably with an inert atomizing gas such as steam, across the flowing stream of catalyst particles to distribute hydrocarbon feed to the reactor riser 26. Upon contacting the hydrocarbon feed with catalyst in the reactor riser 26 the heavier hydrocarbon feed cracks to produce lighter gaseous cracked products while coke is deposited on the catalyst particles to produce spent catalyst.

The resulting mixture of gaseous product hydrocarbons and spent catalyst continues upwardly through the reactor riser 26 and are received in the reactor vessel 28 in which the spent catalyst and gaseous product are separated. Disengaging arms discharge the mixture of gas and catalyst from a top of the reactor riser 26 through outlet ports 36 into a disengaging vessel 38 that effects partial separation of gases from the catalyst. A transport conduit carries the hydrocarbon vapors, stripping media and entrained catalyst to one or more cyclones 42 in the reactor vessel 28 which separates spent catalyst from the hydrocarbon gaseous product stream. Gas conduits deliver separated hydrocarbon cracked gaseous streams from the cyclones 42 to a collection plenum 44 for passage of a cracked product stream to a cracked product line 46 via an outlet nozzle and eventually into the FCC recovery zone 100 for product recovery.

Diplegs discharge catalyst from the cyclones 42 into a lower bed in the reactor vessel 28. The catalyst with adsorbed or entrained hydrocarbons may eventually pass from the lower bed into a stripping section 48 across ports defined in a wall of the disengaging vessel 38. Catalyst separated in the disengaging vessel 38 may pass directly into the stripping section 48 via a bed. A fluidizing distributor delivers inert fluidizing gas, typically steam, to the stripping section 48. The stripping section 48 contains baffles or other equipment to promote contacting between a stripping gas and the catalyst. The stripped spent catalyst leaves the stripping section 48 of the disengaging vessel 38 of the reactor vessel 28 stripped of hydrocarbons. A portion of the spent catalyst, preferably stripped, leaves the disengaging vessel 38 of the reactor vessel 28 through a spent catalyst conduit 50 and passes into the regenerator vessel 30. A second portion of the spent catalyst may be recirculated in recycle conduit 52 from the disengaging vessel 38 back to a base of the riser 26 at a rate regulated by a slide valve to recontact the feed without undergoing regeneration.

The riser 26 can operate at any suitable temperature, and typically operates at a temperature of about 150° to about 580° C. at the riser outlet 36. The pressure of the riser is from about 69 to about 517 kPa (gauge) (10 to 75 psig) but typically less than about 275 kPa (gauge) (40 psig). The catalyst-to-oil ratio, based on the weight of catalyst and feed hydrocarbons entering the riser, may range up to 30:1 but is typically between about 4:1 and about 25:1. Steam may be passed into the reactor riser 26 and reactor vessel 28 at a rate between about 2 and about 7 wt % for maximum gasoline production and about 10 to about 30 wt % for maximum light olefin production. The average residence time of catalyst in the riser may be less than about 5 seconds.

The catalyst in the reactor 22 can be a single catalyst or a mixture of different catalysts. Usually, the catalyst includes two catalysts, namely a first FCC catalyst, and a second FCC catalyst. Such a catalyst mixture is disclosed in, e.g., U.S. Pat. No. 7,312,370 B2. Generally, the first FCC catalyst may include any of the well-known catalysts that are used in the art of FCC. Preferably, the first FCC catalyst includes a large pore zeolite, such as a Y-type zeolite, an active alumina material, a binder material, including either silica or alumina, and an inert filler such as kaolin.

Typically, the zeolites appropriate for the first FCC catalyst have a large average pore size, usually with openings of greater than about 0.7 nm in effective diameter defined by greater than about 10, and typically about 12, member rings. Suitable large pore zeolite components may include synthetic zeolites such as X and Y zeolites, mordenite and faujasite. A portion of the first FCC catalyst, such as the zeolite portion, can have any suitable amount of a rare earth metal or rare earth metal oxide.

The second FCC catalyst may include a medium or smaller pore zeolite catalyst, such as exemplified by at least one of ZSM-5, ZSM-11, ZSM-12, ZSM-23, ZSM-35, ZSM-38, ZSM-48, and other similar materials. Other suitable medium or smaller pore zeolites include ferrierite, and erionite. Preferably, the second component has the medium or smaller pore zeolite dispersed on a matrix including a binder material such as silica or alumina and an inert filler material such as kaolin. These catalysts may have a crystalline zeolite content of about 10 to about 50 wt % or more, and a matrix material content of about 50 to about 90 wt %. Catalysts containing at least about 40 wt % crystalline zeolite material are typical, and those with greater crystalline zeolite content may be used. Generally, medium and smaller pore zeolites are characterized by having an effective pore opening diameter of less than or equal to about 0.7 nm and rings of about 10 or fewer members. Preferably, the second FCC catalyst component is an MFI zeolite having a silicon-to-aluminum ratio greater than about 15. In one exemplary embodiment, the silicon-to-aluminum ratio can be about 15 to about 35.

The total catalyst mixture in the reactor 22 may contain about 1 to about 25 wt % of the second FCC catalyst, including a medium to small pore crystalline zeolite, with greater than or equal to about 7 wt % of the second FCC catalyst being preferred. When the second FCC catalyst contains about 40 wt % crystalline zeolite with the balance being a binder material, an inert filler, such as kaolin, and optionally an active alumina component, the catalyst mixture may contain about 0.4 to about 10 wt % of the medium to small pore crystalline zeolite with a preferred content of at least about 2.8 wt %. The first FCC catalyst may comprise the balance of the catalyst composition. The high concentration of the medium or smaller pore zeolite as the second FCC catalyst of the catalyst mixture can improve selectivity to light olefins. In one exemplary embodiment, the second FCC catalyst can be a ZSM-5 zeolite and the catalyst mixture can include about 0.4 to about 10 wt % ZSM-5 zeolite excluding any other components, such as binder and/or filler.

The regenerator vessel 30 is in downstream communication with the reactor vessel 28. In the regenerator vessel 30, coke is combusted from the portion of spent catalyst delivered to the regenerator vessel 30 by contact with an oxygen-containing gas such as air to regenerate the catalyst. The spent catalyst conduit 50 feeds spent catalyst to the regenerator vessel 30. The spent catalyst from the reactor vessel 28 usually contains carbon in an amount of from 0.2 to 2 wt %, which is present in the form of coke. An oxygen-containing combustion gas, typically air, enters the lower chamber 54 of the regenerator vessel 30 through a conduit and is distributed by a distributor 56. As the combustion gas enters the lower chamber 54, it contacts spent catalyst entering from spent catalyst conduit 50 and lifts the catalyst at a superficial velocity of combustion gas in the lower chamber 54 of perhaps at least 1.1 m/s (3.5 ft/s) under fast fluidized flow conditions. In an embodiment, the lower chamber 54 may have a catalyst density of from 48 to 320 kg/m³ (3 to 20 lb/ft³) and a superficial gas velocity of 1.1 to 2.2 m/s (3.5 to 7 ft/s). The oxygen in the combustion gas contacts the spent catalyst and combusts carbonaceous deposits from the catalyst to at least partially regenerate the catalyst and generate flue gas.

The mixture of catalyst and combustion gas in the lower chamber 54 ascends through a frustoconical transition section to the transport, riser section of the lower chamber 54. The mixture of catalyst particles and flue gas is discharged from an upper portion of the riser section into the upper chamber 60. Substantially completely or partially regenerated catalyst may exit the top of the transport, riser section 58. Discharge is effected through a disengaging device 58 that separates a majority of the regenerated catalyst from the flue gas. The catalyst and gas exit downwardly from the disengaging device 58. The sudden loss of momentum and downward flow reversal cause a majority of the heavier catalyst to fall to the dense catalyst bed and the lighter flue gas and a minor portion of the catalyst still entrained therein to ascend upwardly in the upper chamber 60. Cyclones 62 further separate catalyst from ascending gas and deposits catalyst through dip legs into a dense catalyst bed. Flue gas exits the cyclones 62 through a gas conduit and collects in a plenum 64 for passage to an outlet nozzle of regenerator vessel 30. Catalyst densities in the dense catalyst bed are typically kept within a range of from about 640 to about 960 kg/m³ (40 to 60 lb/ft³).

The regenerator vessel 30 typically has a temperature of about 594° to about 704° C. (1100° to 1300° F.) in the lower chamber 54 and about 649° to about 760° C. (1200° to 1400° F.) in the upper chamber 60. Regenerated catalyst from dense catalyst bed is transported through regenerated catalyst pipe 32 from the regenerator vessel 30 back to the reactor riser 26 through the control valve where it again contacts the feed in line 24 as the FCC process continues. The cracked product stream in the cracked product line 46 from the reactor 22, relatively free of catalyst particles and including the stripping fluid, exit the reactor vessel 28 through an outlet nozzle. The cracked products stream in the line 46 may be subjected to additional treatment to remove fine catalyst particles or to further prepare the stream prior to fractionation. The line 46 transfers the cracked products stream to the FCC recovery zone 100, which is in downstream communication with the FCC zone 20. The FCC recovery zone 100 typically includes a main fractionation column and a gas recovery section. The FCC recovery zone can include many fractionation columns and other separation equipment. The FCC recovery zone 100 can recover a propylene product stream in propylene line 102, a gasoline stream in gasoline line 104, a light olefin stream in light olefin line 106 and an LCO stream in LCO line 107 among others from the cracked product stream in cracked product line 46. The light olefin stream in light olefin line 106 comprises an oligomerization feed stream having C₄ hydrocarbons including C₄ olefins and perhaps having C₅ hydrocarbons including C₅ olefins.

Before cracked products can be fed to the oligomerization zone 130, the light olefin stream in light olefin line 106 may require purification. Many impurities in the light olefin stream in light olefin line 106 can poison an oligomerization catalyst. Carbon dioxide and ammonia can attack acid sites on the catalyst. Sulfur containing compounds, oxygenates, and nitriles can harm oligomerization catalyst. Acetylenes and diolefins can polymerize and produce gums on the catalyst or equipment. Consequently, the light olefin stream which comprises the oligomerization feed stream in light olefin line 106 may be purified in an optional purification zone 110.

The light olefin stream in light olefin line 106 may be introduced into an optional mercaptan extraction unit 112 to remove mercaptans to lower concentrations. In the mercaptan extraction unit 112, the light olefin feed may be prewashed in an optional prewash vessel containing aqueous alkali to convert any hydrogen sulfide to sulfide salt which is soluble in the aqueous alkaline stream. The light olefin stream, now depleted of any hydrogen sulfide, is contacted with a more concentrated aqueous alkali stream in an extractor vessel. Mercaptans in the light olefin stream react with the alkali to yield sodium mercaptides that are soluble in the aqueous alkali phase but not in the hydrocarbon phase. An extracted light olefin stream depleted in mercaptans passes overhead from the extraction column and may be mixed with a solvent that removes COS in route to an optional COS solvent settler. COS may be removed with the solvent from the bottom of the settler, while the overhead light olefin stream may be fed to an optional water wash vessel to remove remaining alkali and produce a sulfur depleted light olefin stream in line 114. The mercaptide rich alkali from the extractor vessel receives an injection of air and a catalyst such as phthalocyanine as it passes from the extractor vessel to an oxidation vessel for regeneration. Oxidizing the mercaptides to disulfides using a catalyst regenerates the alkaline solution. A disulfide separator receives the disulfide rich alkaline from the oxidation vessel. The disulfide separator vents excess air and decants disulfides from the alkaline solution before the regenerated alkaline is drained, washed with oil to remove remaining disulfides and returned to the extractor vessel. Further removal of disulfides from the regenerated alkaline stream is also contemplated. The disulfides may be run through a sand filter and removed from the process. For more information on mercaptan extraction, reference may be made to U.S. Pat. No. 7,326,333 B2.

In order to prevent polymerization and gumming in the oligomerization reactor that can inhibit equipment and catalyst performance, it is desired to minimize diolefins and acetylenes in the light olefin feed in line 114. Diolefin conversion to monoolefin hydrocarbons may be accomplished by selectively hydrogenating the sulfur depleted stream with a conventional selective hydrogenation reactor 116. Hydrogen may be added to the purified light olefin stream in line 118.

The selective hydrogenation catalyst can comprise an alumina support material preferably having a total surface area greater than 150 m²/g, with most of the total pore volume of the catalyst provided by pores with average diameters of greater than 600 angstroms, and containing surface deposits of about 1.0 to 25.0 wt % nickel and about 0.1 to 1.0 wt % sulfur such as disclosed in U.S. Pat. No. 4,695,560. Spheres having a diameter between about 0.4 and 6.4 mm ( 1/64 and ¼ inch) can be made by oil dropping a gelled alumina sol. The alumina sol may be formed by digesting aluminum metal with an aqueous solution of approximately 12 wt % hydrogen chloride to produce an aluminum chloride sol. The nickel component may be added to the catalyst during the sphere formation or by immersing calcined alumina spheres in an aqueous solution of a nickel compound followed by drying, calcining, purging and reducing. The nickel containing alumina spheres may then be sulfided. A palladium catalyst may also be used as the selective hydrogenation catalyst.

The selective hydrogenation process is normally performed at relatively mild hydrogenation conditions. These conditions will normally result in the hydrocarbons being present as liquid phase materials. The reactants will normally be maintained under the minimum pressure sufficient to maintain the reactants as liquid phase hydrocarbons which allow the hydrogen to dissolve into the light olefin feed. A broad range of suitable operating pressures therefore extends from about 276 (40 psig) to about 5516 kPa gauge (800 psig). A relatively moderate temperature between about 25° C. (77° F.) and about 350° C. (662° F.) should be employed. The liquid hourly space velocity of the reactants through the selective hydrogenation catalyst should be above 1.0 hr⁻¹. Preferably, it is between 5.0 and 35.0 hr⁻¹. The molar ratio of hydrogen to diolefinic hydrocarbons may be maintained between 1.5:1 and 2:1. The hydrogenation reactor is preferably a cylindrical fixed bed of catalyst through which the reactants move in a vertical direction.

A purified light olefin stream depleted of sulfur containing compounds, diolefins and acetylenes exits the selective hydrogenation reactor 116 in line 120. The optionally sulfur and diolefin depleted light olefin stream in line 120 may be introduced into an optional nitrile removal unit (NRU) such as a water wash unit 122 to reduce the concentration of oxygenates and nitriles in the light olefin stream in line 120. Water is introduced to the water wash unit in line 124. An oxygenate and nitrile-rich aqueous stream in line 126 leaves the water wash unit 122 and may be further processed. A drier may follow the water wash unit 122. Other NRU's may be used in place of the water wash. A NRU usually comprises a group of regenerable beds that adsorb nitriles and other nitrogen components from the light olefin stream. Examples of NRU's can be found in U.S. Pat. Nos. 4,831,206, 5,120,881 and 5,271,835.

A purified light olefin oligomerization feed stream perhaps depleted of sulfur containing compounds, diolefins and/or oxygenates and nitriles is provided in oligomerization feed stream line 128. The light olefin oligomerization feed stream in line 128 may be obtained from the cracked product stream in line 46, so it may be in downstream communication with the FCC zone 20 and/or the FCC recovery zone 100. The oligomerization feed stream need not be obtained from a cracked FCC product stream but may come from another source such as paraffin dehydrogenation unit or a methanol to olefin unit. The selective hydrogenation reactor 116 is in upstream communication with the oligomerization feed stream line 128. The oligomerization feed stream may comprise C₄ hydrocarbons such as butenes, i.e., C₄ olefins, and butanes. Butenes include normal butenes and isobutene. The oligomerization feed stream in oligomerization feed stream line 128 may comprise C₅ hydrocarbons such as pentenes, i.e., C₅ olefins, and pentanes. Pentenes include normal pentenes and isopentenes. Typically, the oligomerization feed stream will comprise about 20 to about 80 wt % olefins and suitably about 40 to about 75 wt % olefins. In an aspect, about 55 to about 75 wt % of the olefins may be butenes and about 25 to about 45 wt % of the olefins may be pentenes. Up to 10 wt %, suitably 20 wt %, typically 25 wt % and most typically 30 wt % of the oligomerization feed may be C₅ olefins.

The oligomerization feed line 128 feeds the oligomerization feed stream to an oligomerization zone 130 which may be in downstream communication with the FCC recovery zone 100. A first oligomerization reactor zone 140 and a second oligomerization reactor zone 160 may be in downstream communication with the oligomerization feed line 128 and the recycle line 226. The oligomerization feed stream in oligomerization feed line 128 may be mixed with a recycle stream from line 226 prior to entering the oligomerization zone 130 to provide a mixed oligomerization feed stream in a mixed oligomerization feed conduit 132. The mixed oligomerization feed stream may include the recycle stream from recycle line 226 or the recycle stream from recycle line 226 may be fed to the first oligomerization reactor zone 140 and/or the second oligomerization reactor zone 160 separately from the oligomerization feed stream.

The mixed oligomerization feed conduit 132 splits into a first oligomerization feed stream in a first oligomerization feed conduit 133 and a second oligomerization feed stream in a second oligomerization feed conduit 134 at a split 136 to feed the oligomerization feed stream to the first oligomerization reactor zone 140 and the second oligomerization reactor zone 160 in parallel. Additional oligomerization reactor zones can be provided by splitting additional oligomerization feed streams from the mixed oligomerization feed conduit 132 and delivering the additional oligomerization feed streams to respective oligomerization reactors in parallel to the first oligomerization reactor zone 140 and the second oligomerization reactor zone 160. An oligomerization reactor zone can comprise a train of reactors in series, but in parallel with another oligomerization reactor zone.

Preferably, the split 136 may provide the first oligomerization feed stream and the second oligomerization feed stream in aliquot portions of equivalent compositions. Control valves 133′ and 134′ set the proportion of flow to each of the first oligomerization reactor zone 140 and the second oligomerization reactor zone 160.

The first oligomerization reactor zone 140 is in downstream communication with the first oligomerization feed conduit 133 and the recycle conduit 226, and a second oligomerization reactor zone 160 may be in downstream communication with said second oligomerization feed conduit 134 and the recycle conduit 226. A first conduit control valve 133′ is situated on the first oligomerization feed conduit 133 and second conduit control valve 134′ is situated on the second oligomerization feed conduit 134. The control valves can be opened to relatively different degrees to allow full, partial or no flow to each of the first oligomerization reactor zone 140 and the second oligomerization reactor zone 160. The first oligomerization feed conduit 133 bypasses the first oligomerization feed stream around the second oligomerization reactor zone 160, and the second oligomerization feed conduit 134 bypasses the second oligomerization feed stream around the first oligomerization reactor zone 140.

When the first conduit control valve 133′ is opened to any degree it allows the first oligomerization feed stream which may include the recycle stream to flow to the first oligomerization reactor zone 140 containing an oligomerization catalyst and bypassing the first oligomerization feed stream which may include the recycle stream around the second oligomerization reactor zone 160. The first oligomerization feed stream may include a recycle stream from recycle line 226 or a recycle stream from recycle line 226 may be fed to the first oligomerization reactor zone 140 separately from the first oligomerization feed stream in first oligomerization feed line 133.

The oligomerization feed stream in line 132 may comprise about 10 to about 50 wt % olefins and suitably about 25 to about 40 wt % olefins. Accordingly, the oligomerization feed stream may comprise no more than about 38 wt % butene and in another aspect, the oligomerization feed stream may comprise no more than about 23 wt % pentene. The oligomerization feed stream to the oligomerization zone 130 in the oligomerization feed conduit 132 may comprise at least about 10 wt % butene, at least about 5 wt % pentene and preferably no more than about 1 wt % hexene. In a further aspect, the oligomerization feed stream may comprise no more than about 0.1 wt % hexene and no more than about 0.1 wt % propylene. At least about 40 wt % of the butene in the oligomerization feed stream may be normal butene. In an aspect, it may be that no more than about 70 wt % of the oligomerization feed stream is normal butene. At least about 40 wt % of the pentene in the oligomerization feed stream may be normal pentene. In an aspect, no more than about 70 wt % of the oligomerization feed stream in the mixed oligomerization feed conduit 132 may be normal pentene.

The first oligomerization reactor zone 140 comprises a first oligomerization reactor 138. The first oligomerization reactor may be preceded by an optional guard bed for removing catalyst poisons that is not shown. The first oligomerization reactor 138 contains an oligomerization catalyst. An oligomerization feed stream may be preheated before entering the first oligomerization reactor 138 in the oligomerization reactor zone 140. The first oligomerization reactor 138 may contain a first catalyst bed 142 of oligomerization catalyst. The first oligomerization reactor 138 may be an upflow reactor to provide a uniform feed front through the catalyst bed, but other flow arrangements are contemplated. In an aspect, the first oligomerization reactor 138 may contain an additional bed or beds 144 of oligomerization catalyst. The first oligomerization feed stream in line 133 which may include a recycle feed stream from recycle line 226 that is fed to the first oligomerization reactor 138.

C₄ olefins in the first oligomerization feed stream and in the recycle stream oligomerize over the oligomerization catalyst to provide an oligomerate comprising C₄ olefin dimers and trimers. C₅ olefins that may be present in the first and second oligomerization feed stream oligomerize over the oligomerization catalyst to provide an oligomerate comprising C₅ olefin dimers and trimers and co-oligomerize with C₄ olefins to make C₉ olefins. The oligomerization produces other oligomers with additional carbon numbers in the first oligomerization reactor zone 140. Oligomerization effluent from the first bed 142 may optionally be quenched with a liquid such as recycled oligomerate, a portion of the oligomerization feed from the first oligomerization feed conduit 133, or a portion of the recycle stream from the recycle line 226 before entering the additional bed 144. Other means of controlling the reaction exotherm are also envisioned, such as the use of coolers between catalyst beds to remove heat. Oligomerized product, also known as oligomerate, exits the first oligomerization reactor 138 in oligomerate line 146.

It is important that normal butene conversion to butene oligomers be no greater than 50% based on the normal butenes in the first oligomerization feed conduit 133 across the first oligomerization reactor zone 140, which in the embodiment of FIG. 1 is a first oligomerization reactor 138 comprising a first bed 142 and the additional bed 144 and across any additional beds in first oligomerization reactor 138 or in additional oligomerization reactors in the first oligomerization reactor zone 140.

A first oligomerate conduit 146, in communication with the oligomerization reactor zone 140, withdraws a first oligomerate stream from the oligomerization reactor zone 140. The first oligomerate conduit 146 may be in downstream communication with the first oligomerization reactor 138.

When the second conduit control valve 134′ is opened to any degree it allows the second oligomerization feed stream comprising C₄ olefins to flow to the second oligomerization reactor zone 160 containing an oligomerization catalyst and bypassing the second oligomerization feed stream around the first oligomerization reactor zone 140. The composition of the second oligomerization feed stream in the second oligomerization feed line 134 may be the same as in the first oligomerization feed stream in the first oligomerization feed line 133, but the flow rates through each may be varied by adjusting the respective control valves 133′ and 134′. The second oligomerization feed stream may include a recycle stream from recycle line 226 or a recycle stream from recycle line 226 may be fed to the second oligomerization reactor zone 160 separately from the second oligomerization feed stream in line 134.

The second oligomerization reactor zone 160 comprises a second oligomerization reactor 162. A second oligomerization feed stream may be preheated before entering the second oligomerization reactor 162 in the second oligomerization reactor zone 160. The second oligomerization feed stream in line 134 which may include a recycle feed stream is fed to the second oligomerization reactor 162.

The second oligomerization reactor 162 may contain a first catalyst bed 164 of oligomerization catalyst. The second oligomerization reactor 162 may be an upflow reactor to provide a uniform feed front through the catalyst bed, but other flow arrangements are contemplated. In an aspect, the second oligomerization reactor 162 may contain an additional bed or beds 166 of oligomerization catalyst. Oligomerization effluent from the first bed 164 may be quenched with a liquid such as recycled oligomerate, a portion of the oligomerization feed from the second oligomerization feed conduit 134, or a portion of the recycle stream from the recycle line 226 before entering the additional bed 166. Other means of controlling the reaction exotherm are also envisioned, such as the use of coolers between catalyst beds to remove heat. Oligomerized product, also known as oligomerate, exits the second oligomerization reactor 162 in line 168.

A second oligomerate conduit 168 in communication with the second oligomerization reactor zone 160 withdraws a second oligomerate from the second oligomerization reactor zone 160 comprising heavier olefins. The second oligomerate conduit 168 is in downstream communication with the second oligomerization reactor 162.

C₄ olefins in the second oligomerization feed stream and in the recycle stream oligomerize over the oligomerization catalyst to provide an oligomerate comprising C₄ olefin dimers and trimers. C₅ olefins that may be present in the second oligomerization feed stream oligomerize over the oligomerization catalyst to provide an oligomerate comprising C₅ olefin dimers and trimers and co-oligomerize with C₄ olefins to make C₉ olefins. The oligomerization produces other oligomers with additional carbon numbers in the second oligomerization reactor zone 160. It is important that normal butene conversion to butene oligomers be no greater than 50% based on the normal butenes in the second oligomerization feed conduit 134 across the second oligomerization reactor zone 160, which in the embodiment of FIG. 1 is a first oligomerization reactor 162 comprising a first bed 164 and the additional bed 166 and across any additional beds in second oligomerization reactor 162 or in additional oligomerization reactors in the second oligomerization reactor zone 160.

If more than the first oligomerization reactor zone 140 or the second oligomerization reactor zone 160 is used, conversion is achieved over all of the oligomerization reactors zones.

We have found that adding C₅ olefins to the feed to the oligomerization reactor reduces oligomerization to heavier, distillate range material. However, when C₅ olefins dimerize with themselves or co-dimerize with C₄ olefins, the C₉ olefins and C₁₀ olefins produced do not continue to oligomerize as quickly as C₈ olefins produced from C₄ olefin dimerization. Thus, the amount of net gasoline produced can be increased. In addition, the resulting C₉ olefins and C₁₀ olefins in the product have a very high octane value.

The first oligomerization reactor zone 140 and the second oligomerization reactor zone 160 may contain an oligomerization catalyst. The oligomerization catalyst may comprise a zeolitic catalyst. The zeolite may comprise between 5 and 95 wt % of the catalyst. Suitable zeolites include zeolites having a structure from one of the following classes: MFI, MEL, SFV, SVR, ITH, IMF, TUN, FER, EUO, BEA, FAU, BPH, MEI, MSE, MWW, UZM-8, MOR, OFF, MTW, TON, MTT, AFO, ATO, and AEL. These three letter codes for structure types are assigned and maintained by the International Zeolite Association Structure Commission in the ATLAS OF ZEOLITE FRAMEWORK TYPES, which is found at http://www.iza-structure.org/databases/. In a preferred aspect, the oligomerization catalyst may comprise a zeolite with a framework having a ten-ring pore structure. Examples of suitable zeolites having a ten-ring pore structure include those comprising TON, MTT, MFI, MEL, AFO, AEL, EUO and FER. In a further preferred aspect, the oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the oligomerization catalyst comprises an MTT zeolite.

The oligomerization catalyst may be formed by combining the zeolite with a binder, and then forming the catalyst into pellets. The pellets may optionally be treated with a phosphoric reagent to create a zeolite having a phosphorous component between 0.5 and 15 wt % of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present invention is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark Versal. A preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina.

A suitable oligomerization catalyst is prepared by mixing proportionate volumes of zeolite and alumina to achieve the desired zeolite-to-alumina ratio. In an embodiment, about 5 to about 80, typically about 10 to about 60, suitably about 15 to about 40 and preferably about 20 to about 30 wt % MTT zeolite and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize surface acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

The oligomerization reaction conditions in the oligomerization reactors 138, 162 in the oligomerization zone 130 are set to keep the reactant fluids in the liquid phase. With liquid oligomerate recycle, lower pressures are necessary to maintain liquid phase. Operating pressures include between about 2.1 MPa (300 psia) and about 10.5 MPa (1520 psia), suitably at a pressure between about 2.1 MPa (300 psia) and about 6.9 MPa (1000 psia) and preferably at a pressure between about 2.8 MPa (400 psia) and about 4.1 MPa (600 psia). Lower pressures may be suitable if the reaction is kept in the liquid phase.

For the zeolite catalyst, the temperature of the oligomerization reactor zone 140 expressed in terms of a maximum bed temperature is in a range between about 150° and about 300° C. Because gasoline oligomerate is desired, temperatures in the high end of the range are desired to promote back cracking of diesel range oligomerate back down into the gasoline range such as between about 160° and about 240° C. The weight hourly space velocity should be between about 0.5 and about 5 hr⁻¹, preferably at least 2 hr⁻¹. Across a single bed of oligomerization catalyst, the exothermic reaction will cause the temperature to rise. Consequently, the oligomerization reactor may be operated to allow the temperature at the outlet to be over about 25° C. greater than the temperature at the inlet.

We have found that by operating the oligomerization zone 130 at low conversion per pass with a high recycle rate a high yield of gasoline product can be achieved over zeolitic catalyst. We have found that maintaining conversion of normal butene at no more than about 50% per pass through the oligomerization zone and no less than about 10% per pass, a high yield of gasoline can be achieved. Additionally, under these conditions, a reasonably high selectivity of C₈-C₁₁ olefins of at least about 50% can be achieved. To achieve the low conversion per pass, a combined feed ratio (CFR) should be at least about 2 and no more than about 10. Feeding the first oligomerization feed stream and the second oligomerization feed stream through two or more reactor trains in parallel can allow maintenance of a conversion per pass that is relatively low while still achieving adequate overall conversion.

An oligomerization recovery zone 200 is in downstream communication with the oligomerization zone 130 and the first oligomerate conduit 146 and the second oligomerate conduit 168. The combined oligomerate conduit 180 is in downstream communication with the first oligomerate conduit 146 and the second oligomerate conduit 168 and removes the oligomerate stream from the oligomerization zone 130. The combined oligomerate conduit 180 carries the first oligomerate stream from the first oligomerization reactor 138 in the first oligomerate conduit 146 and the second oligomerate stream from the second oligomerization reactor 162 in the second oligomerate conduit 168, and the oligomerate streams are separated together in the oligomerization recovery zone 200.

The oligomerate stream in oligomerate conduit 180 may comprise less than about 60 wt % C₁₂+ hydrocarbons when the C₅ olefins comprise between about 15 and about 50 wt % and preferably between about 20 and about 40 wt % of the olefins in the oligomerization feed. Furthermore, the net gasoline yield may be at least about 40 wt % when C₅ olefins are present in the oligomerization feed.

The oligomerization recovery zone 200 may include a depentanizer column 220 which separates the oligomerate stream between vapor and liquid into a first vaporous oligomerate overhead light stream comprising C₄ and/or C₅ olefins and hydrocarbons in an overhead line 222 and a liquid oligomerate bottom product stream comprising C₆+ olefins and hydrocarbons in a bottom line 224. It is desired to maintain liquid phase in the oligomerization reactors. This is typically achieved by saturating product olefins and recycling them to the oligomerization reactor as a liquid. However, saturating olefins would inactivate unreacted olefins being recycled to the oligomerization zone 130 in the recycle stream. The oligomerization zone 130 can only further oligomerize olefins in the recycle stream. Liquid phase may be maintained in the oligomerization zone 130 by incorporating into the feed a recycle stream from the oligomerization recovery zone 200 comprising C₄ and C₅ hydrocarbons including unreacted olefins. The overhead pressure in the depentanizer column 220 may be between about 50 and about 100 kPa (gauge) and the bottom temperature may be between about 200° and about 275° C.

The light stream in the overhead line 222 may comprise at least 70 wt % and suitably at least 90 wt % C₅ hydrocarbons which can then act as a solvent in the oligomerization zone 130 to maintain liquid phase therein and to provide recycled, unreacted olefins back to the oligomerization reactor. The light stream comprising C₄ and/or C₅ hydrocarbons should have less than 10 wt % C₆ hydrocarbons and preferably less than 1 wt % C₆ hydrocarbons.

The light stream may be condensed and recycled to the oligomerization zone 130 as a recycle stream in a recycle line 226 to maintain the liquid phase in the oligomerization reactors 138, 162 operating in the oligomerization zone 130. The recycle stream may comprise C₄ and/or C₅ olefins that can oligomerize in the oligomerization zone. The C₄ and/or C₅ hydrocarbon presence in the oligomerization zone maintains the oligomerization reactors at liquid phase conditions. The butanes and/or pentanes are easily separated from the heavier olefinic product such as in the depentanizer column 220. The butanes and/or pentanes recycled to the oligomerization zone also dilute the feed olefins to help limit the temperature rise within the reactor due to the exothermicity of the reaction.

In an aspect, the light stream in the overhead line 222 comprising C₄ and C₅ hydrocarbons may be split into a purge stream in purge line 228 and the recycle stream comprising C₄ and C₅ hydrocarbons in the recycle line 226. In an aspect, the recycle stream in the recycle line 226 is taken from the light stream in overhead line 222 is recycled to the oligomerization zone 130 downstream of the selective hydrogenation reactor 116. The light stream in the overhead line 222 and the recycle stream in the recycle line 226 should be understood to be condensed overhead streams. The recycle stream comprising C₄ and C₅ hydrocarbons may be recycled to the oligomerization zone 130 at a mass flow rate which is appropriate to provide a CFR of at least 2 and no more than 10. The recycle rate may be adjusted as necessary to maintain liquid phase in the oligomerization reactors 138 and 162 and to control temperature rise, and to maximize selectivity to gasoline range oligomer products. If the C₄ hydrocarbon content in the recycle line 226 is too high to achieve liquid phase in the oligomerization zone 130, the depentanizer column 220 may be modified to produce an isopentane enriched upper sidecut product stream (not shown) that can be recycled to the oligomerization reactors in addition to, or in replacement of, the recycle stream in recycle line 226 comprised of C₄ and C₅ hydrocarbons. The isopentane rich upper sidecut may require a net purge to avoid buildup of isopentane and an isopentane-rich recycle stream may be delivered through conduit 226.

The purge stream comprising C₄ and/or C₅ hydrocarbons taken from the light stream may be purged from the process in line 228 to avoid C₄ and/or C₅ paraffin build up in the process. The purge stream comprising C₄ and/or C₅ hydrocarbons in line 228 may be subjected to further processing to recover useful components or be blended in the gasoline pool.

Two streams may be taken from the liquid oligomerate bottom product stream in bottom product line 224. A distillate separator feed stream in distillate separator feed line 232 may be taken from the liquid oligomerate bottom product stream in the bottom product line 224. Flow through distillate separator feed line 232 can be regulated by control valve 232′. In a further aspect, a gasoline oligomerate product stream in a gasoline oligomerate product line 250 can be taken from the liquid oligomerate bottom product stream in bottom product line 224. Flow through gasoline oligomerate product line 250 can be regulated by control valve 250′. Flow through distillate separator feed line 232 and gasoline oligomerate product line 250 can be regulated by control valves 232′ and 250′, respectively, such that flow through each line can be shut off or allowed irrespective of the other line.

The liquid oligomerate bottom product stream in bottom product line 224 is a gasoline range material that is highly selective to C₈ to C₁₁ gasoline material. That is, about 40 to about 90 wt % of the resulting liquid oligomerate bottom product stream, for example, in bottom product line 224 is C₈ to C₁₁ material. Consequently, a gasoline oligomerate product stream may be collected from the liquid oligomerate bottom product stream in a gasoline oligomerate product line 250 and blended in the gasoline pool without further treatment such as separation or chemical upgrading. The gasoline oligomerate product line 250 may be in upstream communication with a gasoline tank 252 or a gasoline blending line of a gasoline pool. However, further treatment such as partial or full hydrogenation to reduce olefinicity may be contemplated.

In such a case, control valve 232′ may be all or partially closed and control valve 250′ on oligomerate liquid product line 250 may be opened to allow C₆+ gasoline product to be sent to the gasoline tank 252 or the gasoline blending line.

If it is desired to remove heavier materials from the gasoline product, the oligomerization recovery zone 200 may also include a distillate separator column 240 to which the distillate separator feed stream comprising C₆+ oligomerate hydrocarbons may be fed in distillate separator feed line 232 taken from the liquid oligomerate bottom product stream in line 224 for further separation. The distillate separator column 240 is in downstream communication with the bottom product line 224 of the depentanizer column 220.

At least a portion of the product stream in bottom product line 224 may be split into a gasoline stream and a distillate stream. The distillate separator column 240 separates the distillate separator oligomerate feed stream into an gasoline overhead stream in an overhead line 242 comprising C₆, C₇, C₈, C₉, C₁₀ and/or C₁₁ olefins and a bottom distillate stream comprising C₈+, C₉+, C₁₀+, C₁₁+, or C₁₂+ olefins in a diesel bottom line 244. The overhead pressure in the distillate separator column 240 may be between about 10 and about 60 kPa (gauge) and the bottom temperature may be between about 190° and about 250° C. The bottom temperature can be adjusted between about 175° and about 275° C. to adjust the bottom product between a C₉+ olefin cut and a C₁₂+ olefin cut based on the boiling point range of the gasoline cut desired by the refiner. The diesel bottoms stream in diesel bottoms line 244 may have greater than 30 wt % C₉+ isoolefins.

In an aspect, the gasoline overhead stream in gasoline overhead line 242 may be recovered as product in downstream communication with the recovery zone 200. The gasoline product stream may be subjected to further processing to recover useful components or blended in the gasoline pool. The gasoline product in the gasoline overhead line 242 may be in upstream communication with a gasoline tank 252 or a gasoline blending line of a gasoline pool. In this aspect, the overhead line 242 of the distillate separator column may be in upstream communication with the gasoline tank 252 or the gasoline blending line.

In an aspect, the diesel bottom stream may be recovered as product from the diesel bottom line 244 in downstream communication with the oligomerization recovery zone 200. The diesel product stream may be subjected to further processing to recover useful components or blended in the diesel pool. The diesel bottom line 244 may be in upstream communication with a diesel tank 264 or a diesel blending line of a diesel pool. Additionally, LCO from LCO line 107 may also be blended with diesel in diesel bottom line 244.

The invention will now be further illustrated by the following non-limiting examples.

### EXAMPLES

### Example 1

Feed 1 in Table 1 was contacted with four catalysts to determine their effectiveness in oligomerizing butenes.

TABLE 1

| Component | Fraction, wt % |
|---|---|
| Propylene | 0.1 |
| Iso-C₄'s | 70.04 |
| Isobutylene | 7.7 |
| 1-butene | 5.7 |
| 2-butene (cis and trans) | 16.28 |
| 3-methyl-1-butene | 0.16 |
| Acetone | 0.02 |
| Total | 100 |

Catalyst A is an MTT catalyst purchased from Zeolyst having a product code Z2K019E and extruded with alumina to be 25 wt % zeolite. Of MTT zeolite powder, 53.7 grams was combined with 2.0 grams Methocel and 208.3 grams Catapal B boehmite. These powders were mixed in a muller before a mixture of 18.2 g HNO₃ and 133 grams distilled water was added to the powders. The composition was blended thoroughly in the muller to effect an extrudable dough of about 52% LOI. The dough then was extruded through a die plate to form cylindrical extrudates having a diameter of about 3.18 mm. The extrudates then were air dried, and calcined at a temperature of about 550° C. The MTT catalyst was not selectivated to neutralize surface acid sites such as with an amine.

Catalyst B is a SPA catalyst commercially available from UOP LLC.

Catalyst C is an MTW catalyst with a silica-to-alumina ratio of 36:1. Of MTW zeolite powder made in accordance with the teaching of U.S. Pat. No. 7,525,008 B2, 26.4 grams was combined with and 135.1 grams Versal 251 boehmite. These powders were mixed in a muller before a mixture of 15.2 grams of nitric acid and 65 grams of distilled water were added to the powders. The composition was blended thoroughly in the muller to effect an extrudable dough of about 48% LOI. The dough then was extruded through a die plate to form cylindrical extrudates having a diameter of about 1/32″. The extrudates then were air dried and calcined at a temperature of about 550° C.

Catalyst D is an MFI catalyst purchased from Zeolyst having a product code of CBV-8014 having a silica-to-alumina ratio of 80:1 and extruded with alumina at 25 wt % zeolite. Of MFI-80 zeolite powder, 53.8 grams was combined with 205.5 grams Catapal B boehmite and 2 grams of Methocel. These powders were mixed in a muller before a mixture of 12.1 grams nitric acid and 115.7 grams distilled water were added to the powders. The composition was blended thoroughly in the muller, then an additional 40 grams of water was added to effect an extrudable dough of about 53% LOI. The dough then was extruded through a die plate to form cylindrical extrudates having a diameter of about 3.18 mm. The extrudates then were air dried, and calcined at a temperature of about 550° C.

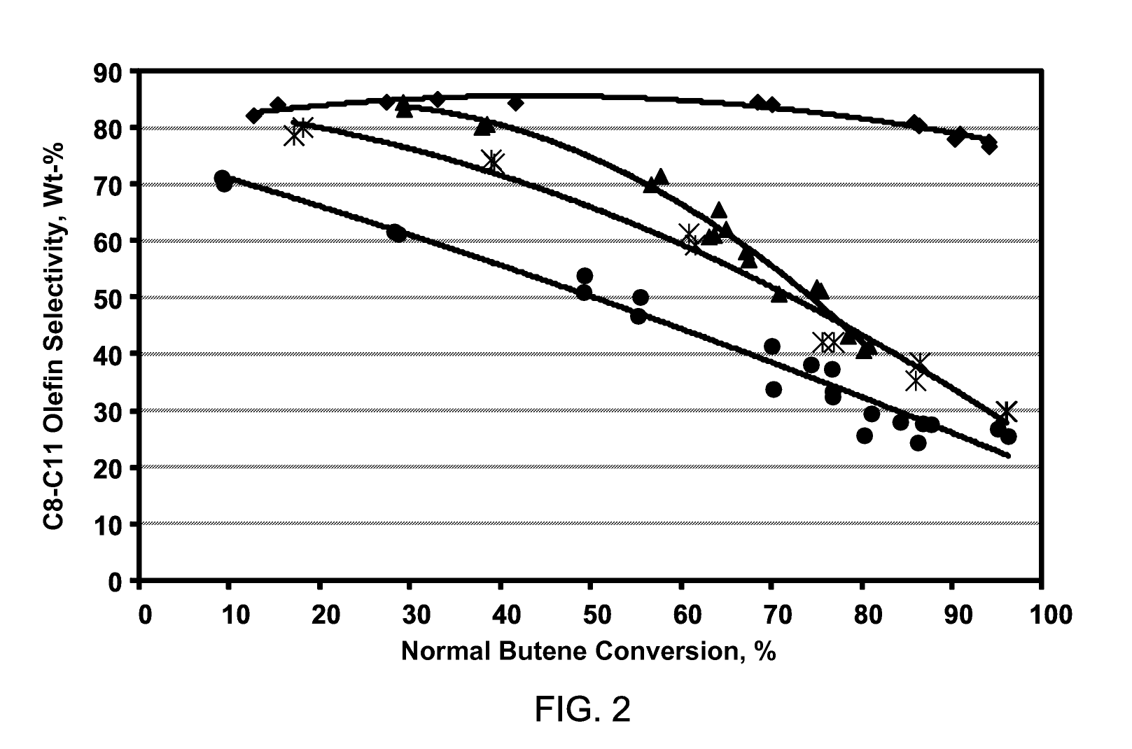

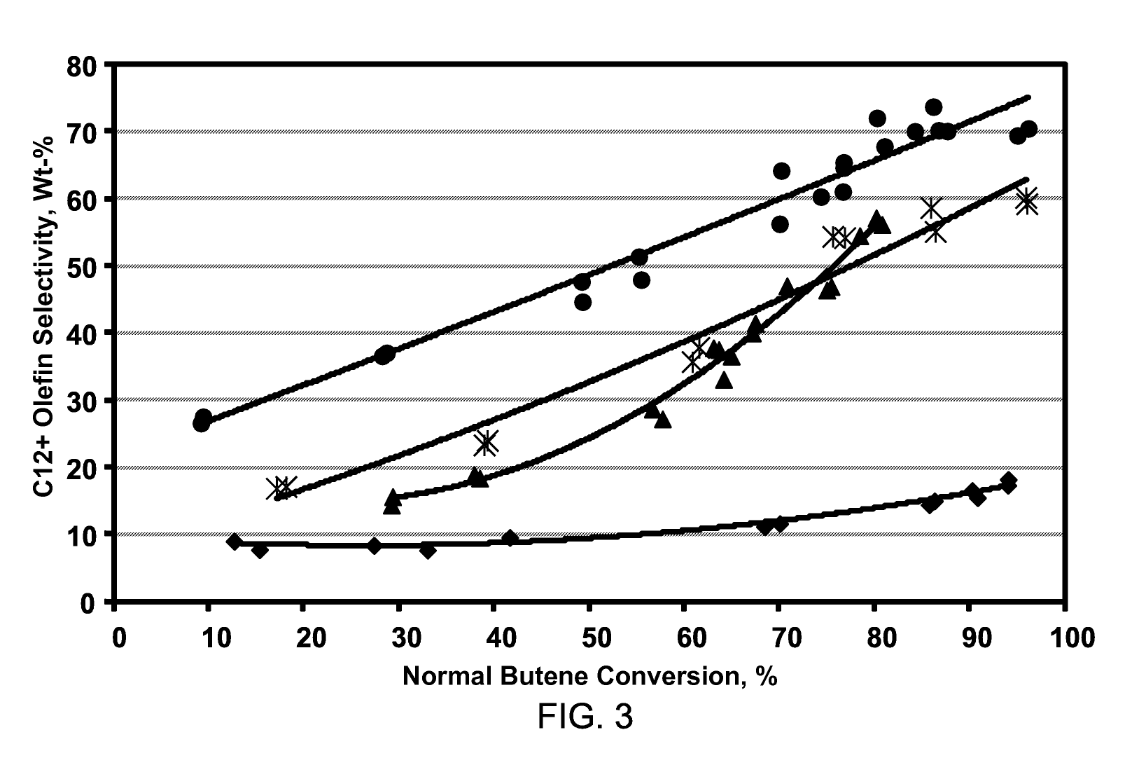

The experiments were operated at 6.2 MPa and inlet temperatures at intervals between 160° and 240° C. to obtain different normal butene conversions. Results are shown in FIGS. 2 and 3. In FIG. 2, C₈ to C₁₁ olefin selectivity is plotted against normal butene conversion to provide profiles for each catalyst. It can be seen that zeolitic catalyst is effective in producing gasoline range material in the C₈-C₁₁ range when normal butene conversion is kept below 50%.

Table 2 compares the RONC ±3 for each product by catalyst and provides a key to FIG. 2. The RONC was determined for the composite product for each catalyst run per ASTM D2699. The SPA catalyst B is superior for selectivity to gasoline-range olefins. The MTT catalyst A is the least effective in producing gasoline range olefins.

TABLE 2

| Catalyst | | | RONC |
|---|---|---|---|
| A | MTT | circles | 92 |
| B | SPA | diamonds | 96 |
| C | MTW | triangles | 97 |
| D | MFI-80 | asterisks | 95 |

The SPA catalyst was able to achieve over 95 wt % yield of gasoline having a RONC of >95 and with an Engler T90 value of 185° C. for the entire product. The T-90 gasoline specification is less than 193° C.

In FIG. 3, C₁₂+ olefin selectivity is plotted against normal butene conversion to provide profiles for each catalyst. Table 3 compares the derived cetane number ±2 for each product by catalyst and provides a key to FIG. 3. The cetane number was determined for the composite product for each catalyst run per ASTM D6890.

TABLE 3

| Catalyst | | | Cetane |
|---|---|---|---|
| A | MTT | circles | 41 |
| B | SPA | diamonds | <14 |
| C | MTW | triangles | 28 |
| D | MFI-80 | asterisks | 36 |

FIG. 3 shows that the MTT catalyst provides the highest C₁₂+ olefin selectivity which reaches over 70 wt %. These selectivities are from a single pass of the feed stream through the oligomerization reactor. Additionally, the MTT catalyst provided C₁₂+ oligomerate with the highest derived cetane. Cetane was derived using ASTM D6890 on the C₁₂+ fraction at the 204° C. (400° F.) cut point.

### Example 2

Two types of feed were oligomerized over oligomerization catalyst A of Example 1, MTT zeolite. Feeds 1 and 2 contacted with catalyst A are shown in Table 4. Feed 1 is from Example 1.

TABLE 4

| | Feed 1 | Feed 2 |
|---|---|---|
| Component | Fraction, wt % | Fraction, wt % |
| propylene | 0.1 | 0.1 |
| isobutane | 70.04 | 9.73 |
| isobutylene | 7.7 | 6.3 |
| 1-butene | 5.7 | 4.9 |
| 2-methyl-2-butene | 0 | 9.0 |
| 2-butene (cis & trans) | 16.28 | 9.8 |
| 3-met-1-butene | 0.16 | 0.16 |
| n-hexane | 0 | 60 |
| acetone | 0.02 | 0.01 |
| Total | 100 | 100 |

In Feed 2, C₅ olefin is made up of 2-methyl-2-butene and 3-methyl-1-butene which comprises 9.16 wt % of the reaction mixture representing about a third of the olefins in the feed. 3-methyl-1-butene is present in both feeds in small amounts. Propylene was present at less than 0.1 wt % in both feeds.

The reaction conditions were 6.2 MPa and a 1.5 WHSV. The maximum catalyst bed temperature was 220° C. Oligomerization achievements are shown in Table 5.

TABLE 5

| | Feed 1 | Feed 2 |
|---|---|---|
| Inlet Temperature, ° C. | 192 | 198 |
| C₄ olefin conversion, % | 98 | 99 |
| nC₄ olefin conversion, % | 97 | 99 |
| C₅ olefin conversion, % | n/a | 95 |
| C₅-C₇ selectivity, wt % | 3 | 5 |
| C₈-C₁₁ selectivity, wt % | 26 | 40 |
| C₁₂-C₁₅ selectivity, wt % | 48 | 40 |
| C₁₆+ selectivity, wt % | 23 | 16 |
| Total C₉+ selectivity, wt % | 78 | 79 |
| Total C₁₂+ selectivity, wt % | 71 | 56 |
| Net gasoline yield, wt % | 35 | 44 |

Normal C₄ olefin conversion reached 99% with C₅ olefins in Feed 2 and was 97 wt % without C₅ olefins in Feed 1. C₅ olefin conversion reached 95%. Feed 2 with C₅ olefins oligomerized to a greater selectivity of lighter, gasoline range product in the C₅-C₇ and C₈-C₁₁ range and a smaller selectivity to heavier distillate range product in the C₁₂-C₁₅ and C₁₆+ range.

By adding C₅ olefins to the feed, a greater yield of gasoline can be made over Catalyst A, MTT. A greater net yield of gasoline and a lower selectivity to C₁₂+ fraction was achieved for Feed 2 than for Feed 1. Gasoline yield was classified by product meeting the Engler T90 requirement and distillate yield was classified by product boiling over 150° C. (300° F.).

### Specific Embodiments

While the following is described in conjunction with specific embodiments, it will be understood that this description is intended to illustrate and not limit the scope of the preceding description and the appended claims.

A first embodiment of the invention is a process for making gasoline comprising feeding an oligomerization feed stream and a recycle stream comprising butenes to an oligomerization zone; and oligomerizing butenes in the oligomerization feed stream and the recycle stream to butene oligomers over a zeolitic catalyst with a combined feed ratio of at least 2 and an n-butene conversion of no more than 50% wherein a selectivity to C₈-C₁₁ olefins is at least about 50%. An embodiment of the invention is one, any or all of prior embodiments in this paragraph up through the first embodiment in this paragraph further comprising oligomerizing pentenes. An embodiment of the invention is one, any or all of prior embodiments in this paragraph up through the first embodiment in this paragraph wherein the zeolitic catalyst is an MTW, MFI or an MTT. An embodiment of the invention is one, any or all of prior embodiments in this paragraph up through the first embodiment in this paragraph wherein the oligomerization temperature is between about 160° and about 240° C. An embodiment of the invention is one, any or all of prior embodiments in this paragraph up through the first embodiment in this paragraph further comprising feeding the oligomerization feed stream and the recycle stream to two oligomerization reactors in parallel. An embodiment of the invention is one, any or all of prior embodiments in this paragraph up through the first embodiment in this paragraph further comprising separating oligomerate streams from the two oligomerization reactors in an oligomerization recovery zone together. An embodiment of the invention is one, any or all of prior embodiments in this paragraph up through the first embodiment in this paragraph further comprising separating a light stream comprising C₄ olefins from an oligomerate stream and recycling a portion of light stream as the recycle stream. An embodiment of the invention is one, any or all of prior embodiments in this paragraph up through the first embodiment in this paragraph further comprising separating a light stream comprising C₅ olefins from the oligomerate stream and recycling a portion of the light stream as the recycle stream. An embodiment of the invention is one, any or all of prior embodiments in this paragraph up through the first embodiment in this paragraph further comprising separating a purge stream from the light stream. An embodiment of the invention is one, any or all of prior embodiments in this paragraph up through the first embodiment in this paragraph further comprising separating a product stream from the light stream. An embodiment of the invention is one, any or all of prior embodiments in this paragraph up through the first embodiment in this paragraph further comprising splitting at least a portion of the product stream into a gasoline stream and a distillate stream.

A second embodiment of the invention is a process for making gasoline comprising oligomerizing an oligomerization feed comprising butenes and a recycle stream comprising butenes over a zeolitic catalyst with a combined feed ratio of at least 2 and an n-butene conversion of no more than 50% to butene oligomers wherein the selectivity to C₈-C₁₁ olefins is at least about 50%; and separating a light stream comprising C₄ olefins from an oligomerate stream and recycling a portion of light stream as the recycle stream. An embodiment of the invention is one, any or all of prior embodiments in this paragraph up through the second embodiment in this paragraph further comprising oligomerizing pentenes. An embodiment of the invention is one, any or all of prior embodiments in this paragraph up through the second embodiment in this paragraph wherein the oligomerization temperature is between about 160° and about 240° C. An embodiment of the invention is one, any or all of prior embodiments in this paragraph up through the second embodiment in this paragraph further comprising feeding the oligomerization feed to two oligomerization reactors in parallel. An embodiment of the invention is one, any or all of prior embodiments in this paragraph up through the second embodiment in this paragraph further comprising separating an oligomerate stream from the two oligomerization reactors together. An embodiment of the invention is one, any or all of prior embodiments in this paragraph up through the second embodiment in this paragraph further comprising separating a light stream comprising C₅ olefins from the oligomerate stream and recycling a portion of the light stream as the recycle stream.

A third embodiment of the invention is a process for making gasoline comprising oligomerizing butenes in an oligomerization feed stream and a recycle stream over a zeolitic catalyst in two oligomerization reactors in parallel with a combined feed ratio of at least 2 and an n-butene conversion of no more than 50% to butene oligomers wherein the selectivity to C₈-C₁₁ olefins is at least about 50%. An embodiment of the invention is one, any or all of prior embodiments in this paragraph up through the third embodiment in this paragraph further comprising separating a light stream comprising C₄ olefins from an oligomerate stream and recycling a portion of light stream as the recycle stream. An embodiment of the invention is one, any or all of prior embodiments in this paragraph up through the third embodiment in this paragraph further comprising separating an oligomerate stream from the two oligomerization reactors in an oligomerization recovery zone together.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process for making gasoline comprising feeding an oligomerization feed stream and a recycle stream comprising butenes to an oligomerization zone; and
oligomerizing butenes in the oligomerization feed stream and the recycle stream to butene oligomers over a zeolitic catalyst with a combined feed ratio of at least 2 and an n-butene conversion of no more than 50% wherein a selectivity to C₈-C₁₁ olefins is at least about 50%.

2. The process of claim 1 further comprising oligomerizing pentenes.

3. The process of claim 1 wherein said zeolitic catalyst is an MTW, MFI or an MTT.

4. The process of claim 1 wherein said oligomerization temperature is between about 160° and about 240° C.

5. The process of claim 1 further comprising feeding said oligomerization feed stream and said recycle stream to two oligomerization reactors in parallel.

6. The process of claim 5 further comprising separating oligomerate streams from said two oligomerization reactors in an oligomerization recovery zone together.

7. The process of claim 1 further comprising separating a light stream comprising C₄ olefins from an oligomerate stream and recycling a portion of light stream as said recycle stream.

8. The process of claim 1 further comprising separating a light stream comprising C₅ olefins from said oligomerate stream and recycling a portion of said light stream as said recycle stream.

9. The process of claim 8 further comprising separating a purge stream from said light stream.

10. The process of claim 8 further comprising separating a product stream from said light stream.

11. The process of claim 10 further comprising splitting at least a portion of said product stream into a gasoline stream and a distillate stream.

12. A process for making gasoline comprising oligomerizing an oligomerization feed comprising butenes and a recycle stream comprising butenes over a zeolitic catalyst with a combined feed ratio of at least 2 and an n-butene conversion of no more than 50% to butene oligomers wherein said selectivity to C₈-C₁₁ olefins is at least about 50%; and separating a light stream comprising C₄ olefins from an oligomerate stream and recycling a portion of light stream as said recycle stream.

13. The process of claim 12 further comprising oligomerizing pentenes.

14. The process of claim 12 wherein said oligomerization temperature is between about 160° and about 240° C.

15. The process of claim 12 further comprising feeding said oligomerization feed to two oligomerization reactors in parallel.

16. The process of claim 15 further comprising separating an oligomerate stream from said two oligomerization reactors together.

17. The process of claim 12 further comprising separating a light stream comprising C₅ olefins from said oligomerate stream and recycling a portion of said light stream as said recycle stream.

18. A process for making gasoline comprising oligomerizing butenes in an oligomerization feed stream and a recycle stream over a zeolitic catalyst in two oligomerization reactors in parallel with a combined feed ratio of at least 2 and an n-butene conversion of no more than 50% to butene oligomers wherein said selectivity to C₈-C₁₁ olefins is at least about 50%.

19. The process of claim 18 further comprising separating a light stream comprising C₄ olefins from an oligomerate stream and recycling a portion of light stream as said recycle stream.

20. The process of claim 19 further comprising separating an oligomerate stream from said two oligomerization reactors in an oligomerization recovery zone together.
